(19) United States
(12) Patent Application Publication
(10) Pub. No.: US 2024/0158522 A1
(43) Pub. Date: May 16, 2024

(54) Methods of Treating Ulcerative Colitis with Anti-LIGHT Antibodies

(71) Applicant: Avalo Therapeutics, Inc., Rockville, MD (US)

(72) Inventor: Garry A. Neil, Havertown, PA (US)

(73) Assignee: Avalo Therapeutics, Inc., Rockville, MD (US)

(21) Appl. No.: 18/421,576

(22) Filed: Jan. 24, 2024

Related U.S. Application Data
(63) Continuation of application No. PCT/US22/74106, filed on Jul. 25, 2022.
(60) Provisional application No. 63/225,675, filed on Jul. 26, 2021.

(51) Int. Cl. C07K 16/28; A61K 39/00

(52) U.S. Cl. CPC C07K 16/2875; A61K 2039/505; A61K 2039/545; C07K 2317/565

(57) ABSTRACT

The present disclosure relates to methods of treating ulcerative colitis with anti-LIGHT antibodies. The disclosure also relates to assaying free LIGHT prior to, during, or after administration of an anti-LIGHT antibody to treat ulcerative colitis.

### CROSS-REFERENCE TO RELATED APPLICATIONS

This application is a continuation of International Patent Application No. PCT/US2022/074106, filed Jul. 25, 2022, which claims the benefit of priority of U.S. Provisional Application No. 63/225,675, filed Jul. 26, 2021, the contents of each of which are incorporated by reference herein in their entireties for any purpose.

### SEQUENCE LISTING

The instant application contains a Sequence Listing which has been submitted electronically in XML format and is hereby incorporated by reference in its entirety. Said XML copy, created on Jul. 6, 2022, is named “01118-0054-00PCT ST26.xml” and is 109,180 bytes in size.

### FIELD OF THE INVENTION

The present disclosure relates to methods of treating ulcerative colitis with anti-LIGHT antibodies. The disclosure also relates to assaying free LIGHT prior to, during, or after administration of an anti-LIGHT antibody to treat ulcerative colitis.

### BACKGROUND OF THE INVENTION

There are two primary forms of inflammatory bowel diseases (IBD): Crohn's Disease (CD) and ulcerative colitis (UC). (Wang et al., The critical role of LIGHT in promoting intestinal inflammation and Crohn's disease, J. Immunol., 2005:174;8173-82). IBD including CD and UC are characterized by a chronic relapsing inflammation of the gastrointestinal tract. Id.

UC most commonly presents with blood in the stool and diarrhea. (Ungaro et al., Ulcerative colitis, Lancet, 2017:389;1756-1770). Symptoms can include urgency, incontinence, fatigue, increased frequency of bowel movements, mucus discharge, nocturnal defecations, and abdominal discomfort. Id. The primary goal of treatment is to induce and maintain remission with the long-term goals of preventing disability, colectomy, and colorectal cancer. Id. Potential targets for remission are resolution of clinical symptoms, defined as cessation of rectal bleeding and improvement in bowel habits, and endoscopic healing. Id.

Multiple factors, including genetics, environmental and luminal factors, and mucosal immune dysregulation, have been suggested to play a role in UC pathogenesis. Id. The incidence and prevalence of ulcerative colitis have been increasing over time worldwide. Id. There is a great need for new and improved methods of treating ulcerative colitis.

LIGHT (acronym for “homologous to jymphotoxin, exhibits Inducible expression and competes with HSV Glycoprotein D for binding to HVEM (herpesvirus entry mediator), a receptor expressed on T lymphocytes”), also known as TNF SF14 (tumor necrosis factor superfamily member 14) is an important regulatory cytokine.

LIGHT (TNF SF 14) belongs to the tumor necrosis factor superfamily and is expressed by activated T cells, monocytes-macrophages and additional types of antigen presenting cells. LIGHT is considered one of the “Master Regulators” of the immune system and has a key role in the communication system which controls immune response. LIGHT has a dual mechanism of action; exerting its effects by activating both T cells and B cells as well as upregulating other inflammatory cytokines.

LIGHT activates two key receptors, herpesvirus entry mediator (HVEM) and lymphotoxin β receptor (LTβR), both expressed on lung epithelial cells. Early in infection LIGHT released from neutrophils and macrophages bind cellular receptors, which causes inflammatory cell infiltration, releasing high level of TNF and additional pro-inflammatory cytokines. LIGHT also has a co-stimulatory role in T cell activation driving proinflammatory and tissue damaging effects. (Ware, C. F. Advances in experimental medicine and biology 647, 146-155 (2009); Ware, C. F. Immunological reviews 223, 186-201 (2008)). Therefore, LIGHT has roles in many immune-mediated pathologies such as Crohn's Disease, IBD, Rheumatoid arthritis, and fibrosis. An additional receptor for LIGHT is a decoy receptor (coined DCR3), which binds LIGHT and interferes with its activity by competing with receptor binding. (Steinberg, M. W., et al., M. Seminars in immunopathology 31, 207-221 (2009); Wroblewski, V. J. et al. Biochemical pharmacology 65, 657-667 (2003)). In hyper inflammation and cytokine storm conditions, DcR3 is likely to be overwhelmed, generating high DCR3-free (active) LIGHT.

LIGHT has a role as an important mediator in mucosal inflammation and inflammatory bowel disease (IBD) pathogenesis (Cohavy et al., LIGHT expression by mucosal T cells may regulate IFN-gamma expression in the intestine, J. Immunol., 2004; 173(1):251-8; Ware, CF, Network Communications: Lymphotoxins, LIGHT and TNF, Annual Rev. Immunol., 2005: 23:787-819; Cohavy et al., LIGHT is constitutively expressed on T and NK cells in the human gut and can be induced by CD2-mediated signaling, J. Immunol., 2005:174:646-53; Wang et al., The critical role of LIGHT in promoting intestinal inflammation and Crohn's disease, J. Immunol., 2005:174;8173-82). The human LIGHT gene maps to chromosome 19p13.3, a region that has been implicated in the pathogenesis of IBD. Id. The concept that LIGHT provides a critical pro-inflammatory signal during cellular immune responses is reinforced by studies in IBD patients. LIGHT messenger ribonucleic acid (RNA) is upregulated in biopsies from inflamed areas of small bowel (Cohavy et al., LIGHT is constitutively expressed on T and NK cells in the human gut and can be induced by CD2-mediated signaling, J. Immunol., 2005:174:646-53). In addition, LIGHT is present in elevated levels in UC subjects compared with healthy subjects. (Moraes et al., Systemic inflammatory protein profiles distinguish Irritable Bowel Syndrome (IBS) and Ulcerative Colitis, irrespective of inflammation or IBS-like symptoms, Inflammatory Bowel Disease, 2020:26:874-884). Further, LIGHT overexpression increases intestinal inflammation in mice and anti-LIGHT antibodies have been shown to reduce signs of intestinal inflammation in a mouse model of ulcerative colitis. (Jungbeck et al., Neutralization of LIGHT ameliorates acute dextran sodium sulphate-induced intestinal inflammation, Immunology, 2009:128:451-58; Shaikh et al., Constitutive expression of LIGHT on T cells leads to lymphocyte activation, inflammation, and tissue destruction, J. Immunol., 2001:167:6330-6337). Knockout of LIGHT (or its ligand, HVEM) gene results in reduced intestinal inflammation in some models. (Schaer et al., HVEM Signalling Promotes Colitis, PLoS One, 2011:6(4):e18495).

Decoy receptor 3 belongs to the TNF superfamily (TNFRSF6B) (Yu et al., A newly identified member of tumor necrosis factor receptor superfamily (TR6) suppresses LIGHT-mediated apoptosis, J. Biol. Chem., 1999: 274(20):13733-6). It acts as a decoy receptor that competes with death receptors for ligand binding and is postulated to play a regulatory role in suppressing Fas ligand (FasL)—and LIGHT-mediated cell death and T cell activation as well as to induce angiogenesis via neutralization of TNF-like ligand 1A (TL1A) (Yu et al. 1999). Defective variants of DcR3 have recently been observed in patients with pediatric onset IBD which further suggests an important protective role for DcR3 (Cardinale et al., Targeted resequencing identifies defective variants of decoy receptor 3 in pediatric-onset inflammatory bowel disease, Genes Immun. 2013: Oct;14(7):447-52), potentially by moderating the effects of TNF and LIGHT.

The roles of LIGHT and DcR3 in the pathogenesis of IBD, described above, provide a rationale for the study of an anti-LIGHT monoclonal antibody in UC patients with or without loss-of-function mutations in DcR3.

### SUMMARY OF THE INVENTION

The present disclosure includes, for example, any one or a combination of the following embodiments:
Embodiment 1. A method of treating ulcerative colitis or an inflammatory condition associated with ulcerative colitis, comprising administering an anti-LIGHT antibody to a subject in need thereof, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences selected from:
   a) SEQ ID NOs: 2, 3, 4, 5, 6, and 7;
   b) SEQ ID NOs: 10, 11, 12, 13, 14, and 15;
   c) SEQ ID NOs: 16, 17, 18, 19, 20, and 21;
   d) SEQ ID NOs: 22, 23, 24, 25, 26, and 27;
   e) SEQ ID NOs: 28, 29, 30, 31, 32, and 33;
   f) SEQ ID NOs: 34, 35, 36, 37, 38, and 39;
   g) SEQ ID NOs: 40, 41, 42, 43, 44, and 45;
   h) SEQ ID NOs: 46, 47, 48, 49, 50, and 51; and
   i) SEQ ID NOs: 52, 53, 54, 55, 56, and 57.
Embodiment 2. A method of treating ulcerative colitis or an inflammatory condition associated with ulcerative colitis, comprising administering an anti-LIGHT antibody to a subject in need thereof, wherein the subject in need thereof has failed treatment with an approved therapeutic dose of an anti-TNFα monoclonal antibody with either no initial response or an initial response to induction with subsequent lost response, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences selected from:
   a) SEQ ID NOs: 2, 3, 4, 5, 6, and 7;
   b) SEQ ID NOs: 10, 11, 12, 13, 14, and 15;
   c) SEQ ID NOs: 16, 17, 18, 19, 20, and 21;
   d) SEQ ID NOs: 22, 23, 24, 25, 26, and 27;
   e) SEQ ID NOs: 28, 29, 30, 31, 32, and 33;
   f) SEQ ID NOs: 34, 35, 36, 37, 38, and 39;
   g) SEQ ID NOs: 40, 41, 42, 43, 44, and 45;
   h) SEQ ID NOs: 46, 47, 48, 49, 50, and 51; and
   i) SEQ ID NOs: 52, 53, 54, 55, 56, and 57.
Embodiment 3. A method of treating ulcerative colitis or an inflammatory condition associated with ulcerative colitis, comprising administering an anti-LIGHT antibody to a subject in need thereof, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences selected from:
   a) SEQ ID NOs: 2, 3, 4, 5, 6, and 7;
   b) SEQ ID NOs: 10, 11, 12, 13, 14, and 15;
   c) SEQ ID NOs: 16, 17, 18, 19, 20, and 21;
   d) SEQ ID NOs: 22, 23, 24, 25, 26, and 27;
   e) SEQ ID NOs: 28, 29, 30, 31, 32, and 33;
   f) SEQ ID NOs: 34, 35, 36, 37, 38, and 39;
   g) SEQ ID NOs: 40, 41, 42, 43, 44, and 45;
   h) SEQ ID NOs: 46, 47, 48, 49, 50, and 51; and
   i) SEQ ID NOs: 52, 53, 54, 55, 56, and 57;

wherein the anti-LIGHT antibody is administered at a dose of 1.0 mg/kg or 3.0 mg/kg every 14 days.
Embodiment 4. A method of treating ulcerative colitis or an inflammatory condition associated with ulcerative colitis, comprising administering an anti-LIGHT antibody to a subject in need thereof, wherein the subject in need thereof has failed treatment with an approved therapeutic dose of an anti-TNFα monoclonal antibody with either no initial response or an initial response to induction with subsequent lost response, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences selected from:
   a) SEQ ID NOs: 2, 3, 4, 5, 6, and 7;
   b) SEQ ID NOs: 10, 11, 12, 13, 14, and 15;
   c) SEQ ID NOs: 16, 17, 18, 19, 20, and 21;
   d) SEQ ID NOs: 22, 23, 24, 25, 26, and 27;
   e) SEQ ID NOs: 28, 29, 30, 31, 32, and 33;
   f) SEQ ID NOs: 34, 35, 36, 37, 38, and 39;
   g) SEQ ID NOs: 40, 41, 42, 43, 44, and 45;
   h) SEQ ID NOs: 46, 47, 48, 49, 50, and 51; and
   i) SEQ ID NOs: 52, 53, 54, 55, 56, and 57;

wherein the anti-LIGHT antibody is administered at a dose of 1.0 mg/kg or 3.0 mg/kg every 14 days.
Embodiment 5. The method of any one of the preceding embodiments, wherein the anti-LIGHT antibody is administered subcutaneously.
Embodiment 6. The method of any one of the preceding embodiments, wherein the method further comprises assaying free LIGHT prior to, during, or after administration of the anti-LIGHT antibody.
Embodiment 7. The method of any one of the preceding embodiments, wherein the subject has elevated free LIGHT.
Embodiment 8. The method of any one of the preceding embodiments, wherein the subject is human.
Embodiment 9. The method of any one of the preceding embodiments, wherein the subject is an adult.
Embodiment 10. The method of any one of the preceding embodiments, wherein the subject is a pediatric subject.
Embodiment 11. The method of any one of the preceding embodiments, wherein the antibody comprises a variable heavy chain (VH) comprising an amino acid sequence of SEQ ID NO: 84.
Embodiment 12. The method of any one of the preceding embodiments, wherein the antibody comprises a variable light chain (VL) comprising an amino acid sequence of SEQ ID NO: 85.
Embodiment 13. The method of any one of the preceding embodiments, wherein the antibody comprises a heavy chain comprising an amino acid sequence of SEQ ID NO: 8.
Embodiment 14. The method of any one of the preceding embodiments, wherein the antibody comprises a light chain comprising an amino acid sequence of SEQ ID NO: 9.
Embodiment 15. The method of any one of embodiments 1-14, wherein administration of the anti-LIGHT antibody decreases the subject's endoscopic Mayo score.
Embodiment 16. The method of any one of embodiments 1-15, wherein administration of the anti-LIGHT antibody decreases the subject's total Mayo score.
Embodiment 17. The method of any one of embodiments 1-16, wherein administration of the anti-LIGHT antibody increases the subject's IBD-Q score.
Embodiment 18. The method of any one of the preceding embodiments, wherein administration of the anti-LIGHT antibody reduces serum free LIGHT in the subject.

### DETAILED DESCRIPTION OF THE INVENTION

The following definitions are provided to facilitate an understanding of the invention. They are not intended to limit the invention in any way.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an “isolated,” or “biologically pure” molecule is a compound that has been removed from its natural milieu. As such, the terms “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“LIGHT” or “TNF SF 14” herein refers to a specific member protein of the tumor necrosis factor superfamily that is expressed by activated T cells, monocytes-macrophages and additional types of antigen presenting cells. “LIGHT” is an acronym for “homologous to Lymphotoxin, exhibits Inducible expression and competes with HSV Glycoprotein D for binding to HVEM (herpesvirus entry mediator), a receptor expressed on T lymphocytes.”

“Free LIGHT” or “free (active) LIGHT” herein refers to non-bound form LIGHT (e.g., LIGHT bound to DcR3), which is the active form of LIGHT. In humans, free LIGHT is neutralized (inactivated) by DcR3, a unique soluble member of the TNFR superfamily, which binds LIGHT in high affinity and inhibits its interactions with two TNF receptors, HVEM and LTPR. “Bound LIGHT,” or the like, refers to LIGHT that is bound to a natural ligand, optionally wherein the natural ligand is HVEM, LTPR, or DcR3. “Total LIGHT,” or the like, refers to the total amount of free LIGHT and bound LIGHT.

“Elevated free LIGHT” as used herein refers to a level of free LIGHT detected in a subject that is higher than a normal control. The normal control can be determined by those of skill in the art as applicable to the particular situation. In some instances, the normal control is an industry standard agreed upon by those of skill as being a level or range of levels that is typical of an individual without a LIGHT-associated condition. In some instances, the normal control is a reference level of LIGHT from the same individual taken at a time point, and whether the subject has elevated LIGHT is determined based on a sample from that same individual taken at a different, typically later, time point.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab)₂, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Sample” or “subject sample” or “biological sample” generally refers to a sample which may be tested for a particular molecule. Samples may include but are not limited to cells, body fluids, including blood, serum, plasma, urine, saliva, stool, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins. Agents are evaluated for potential biological activity by inclusion in screening assays described hereinbelow.

A “subject” can be mammalian. In any of the embodiments involving a subject, the subject can be human. In any of the embodiments involving a subject, the subject can be a cow, pig, monkey, sheep, dog, cat, fish, or poultry.

A “pediatric” subject herein is a human of less than 18 years of age, whereas an “adult” subject is 18 years or older.

“Treatment” or “treat” refers to both therapeutic treatment and prophylactic or preventative measures. Those in need of treatment include those already with the disorder as well as those prone to have the disorder or those in which the disorder is to be prevented. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

Methods of Treating Ulcerative Colitis with Anti-LIGHT Antibody

In some embodiments, a method of treating subjects having ulcerative colitis or an inflammatory condition associated with ulcerative colitis is provided comprising administering an anti-LIGHT antibody to a subject in need thereof. In some embodiments, the subject has failed treatment with an approved therapeutic dose of an anti-TNFα monoclonal antibody with either no initial response or an initial response to induction with subsequent lost response. In some embodiments, the anti-LIGHT antibody is administered at a dose of from about 1.0 mg/kg to about 3.0 mg/kg every 14 days. In some embodiments, the anti-LIGHT antibody is administered as a dose of 1.0 mg/kg every 14 days. In some embodiments, the anti-LIGHT antibody is administered as a dose of 3.0 mg/kg every 14 days. In some embodiments, the anti-LIGHT antibody is administered subcutaneously. In some embodiments, the method further comprises assaying free LIGHT prior to, during, or after administration of the anti-LIGHT antibody. In some embodiments, the subject has elevated free LIGHT. In some embodiments, the subject is human. In some embodiments, the subject is an adult. In some embodiments, the subject is a pediatric subject.

In some embodiments, administration of the anti-LIGHT antibody reduces serum free LIGHT in the subject.

The Mayo Score is the most well-known disease activity instrument for UC. It is a composite instrument scored on a scale from 0 to 12 and includes stool frequency (score from 0 to 3), rectal bleeding (score from 0 to 3), a physician's global assessment (score from 0 to 3), and a sigmoidoscopic/endoscopic evaluation (score from 0 to 3). (Peyrin-Biroulet et al., Defining disease severity in inflammatory bowel diseases: current and future directions, Clinical Gastroenterology and Hepatology 2016:14:348-354; Lewis et al., Use of the non-invasive components of the Mayo Score to assess clinical response in ulcerative colitis, Inflammatory Bowel Disease, 2008:14(12):1660-1666). Portions of the Mayo Score may be used alone, such as the endoscopic Mayo Score. (Ungaro et al., Ulcerative colitis, Lancet, 2017:389;1756-1770).

A patient reported outcome is a measurement derived directly from a patient about any aspect of their health status and has the potential to become a treatment endpoint for UC. Several scales have been used to assess patients' perspectives towards the disease, including the Inflammatory Bowel Disease Questionnaire (IBD-Q). (Peyrin-Biroulet 2016).

In some embodiments, administration of the anti-LIGHT antibody reduces the subject's endoscopic Mayo score, compared to the subject's endoscopic Mayo score prior to administration of the anti-LIGHT antibody or compared to subjects who are not administered the anti-LIGHT antibody. In some embodiments, administration of the anti-LIGHT antibody reduces the subject's total Mayo score, compared to the subject's total Mayo score prior to administration of the anti-LIGHT antibody or compared to subjects who are not administered the anti-LIGHT antibody

In some embodiments, administration of the anti-LIGHT antibody increases the subject's IBD-Q score, for example, compared to the subject's IBD-Q score prior to administration of the anti-LIGHT antibody or compared to subjects who are not administered the anti-LIGHT antibody. In some embodiments, administration of the anti-LIGHT antibody results in the subject's IBD-Q score of 170 or higher. In some embodiments, administration of the anti-LIGHT antibody results in an increase in the subject's IBD-Q score of at least 16 points. In some embodiments, administration of the anti-LIGHT antibody results in an increase in the subject's IBD-Q score of at least 32 points.

### Anti-LIGHT Antibodies

In some embodiments, an anti-LIGHT antibody is utilized for both detection/diagnostic and therapeutic purposes, as well as in the assays described herein. The anti-LIGHT antibody used for detection or diagnostic purposes may be different or the same as the antibody used for therapeutic purposes (even in the same subject).

In some embodiments, the anti-LIGHT antibody useful for therapeutic purposes may comprise the CDR sequences of the E1, E13, E63, F19, or F23 antibodies, which are provided in WO 2008/027338 and U.S. Pat. No. 8,058,402 B2, U.S. Pat. No. 8,461,307 B2, and U.S. Pat. No. 8,974,787 B2, each of which is incorporated herein by reference. In some embodiments, the anti-LIGHT antibody useful for detection/diagnostic purposes is not that same as that which is used for therapeutic purposes.

In some embodiments, the anti-LIGHT antibody comprises a heavy chain comprising three CDR sequences comprising each of SEQ ID NOs: 2, 3, and 4. In some embodiments, the anti-LIGHT antibody comprises a light chain comprising three CDR sequences comprising each of SEQ ID NOs: 5, 6, and 7. In some embodiments, the antibody comprises a heavy chain and a light chain, wherein the heavy chain comprises three CDR sequences comprising each of SEQ ID NOs: 2, 3, and 4, and wherein the light chain comprises three CDR sequences comprising each of SEQ ID NOs: 5, 6, and 7.

In some embodiments, the anti-LIGHT antibody comprises a heavy chain variable region sequence comprising SEQ ID NO: 84 or a sequence having at least 85%, at least 90%, at least 95%, at least 98%, or at least 99% identity to SEQ ID NO: 84. In some embodiments, the anti-LIGHT antibody comprises a light chain variable region sequence comprising SEQ ID NO: 85 or a sequence having at least 85%, at least 90%, at least 95%, at least 98%, or at least 99% identical to SEQ ID NO: 85. In some embodiments, the anti-LIGHT antibody comprises a heavy chain variable region sequence comprising SEQ ID NO: 84 or a sequence having at least 85%, at least 90%, at least 95%, at least 98%, or at least 99% identity to SEQ ID NO: 84, and a light chain variable region sequence comprising SEQ ID NO: 85 or a sequence having at least 85%, at least 90%, at least 95%, at least 98%, or at least 99% identical to SEQ ID NO: 85.

In some embodiments, the anti-LIGHT antibody comprises a heavy chain sequence comprising SEQ ID NO: 8 or a sequence having at least 85%, at least 90%, at least 95%, at least 98%, or at least 99% identity to SEQ ID NO:8. In some embodiments, the anti-LIGHT antibody comprises a light chain sequence comprising SEQ ID NO: 9 or a sequence having at least 85%, at least 90%, at least 95%, at least 98%, or at least 99% identical to SEQ ID NO: 9. In some embodiments, the anti-LIGHT antibody comprises both a heavy chain comprising SEQ ID NO: 8 or a sequence that is at least 85%, at least 90%, at least 95%, at least 98%, or at least 99% identical to SEQ ID NO:8 and a light chain comprising SEQ ID NO: 9 or a sequence that is at least 85%, at least 90%, at least 95%, at least 98%, or at least 99% identical to SEQ ID NO:9.

In some embodiments, the anti-LIGHT antibody comprises a heavy chain comprising three CDR sequences comprising each of SEQ ID NOs: 10, 11, and 12. In some embodiments, the anti-LIGHT antibody comprises a light chain comprising three CDR sequences comprising each of SEQ ID NOs: 13, 14, and 15. In some embodiments, the antibody comprises a heavy chain and a light chain, wherein the heavy chain comprises three CDR sequences comprising each of SEQ ID NOs: 10, 11, and 12, and wherein the light chain comprises three CDR sequences comprising each of SEQ ID NOs: 13, 14, and 15.

In some embodiments, the anti-LIGHT antibody comprises a heavy chain comprising three CDR sequences comprising each of SEQ ID NOs: 16, 17, and 18. In some embodiments, the anti-LIGHT antibody comprises a light chain comprising three CDR sequences comprising each of SEQ ID NOs: 19, 20, and 21. In some embodiments, the antibody comprises a heavy chain and a light chain, wherein the heavy chain comprises three CDR sequences comprising each of SEQ ID NOs: 16, 17, and 18, and wherein the light chain comprises three CDR sequences comprising each of SEQ ID NOs: 19, 20, and 21.

In some embodiments, the anti-LIGHT antibody comprises a heavy chain comprising three CDR sequences comprising each of SEQ ID NOs: 22, 23, and 24. In some embodiments, the anti-LIGHT antibody comprises a light chain comprising three CDR sequences comprising each of SEQ ID NOs: 25, 26, and 27. In some embodiments, the antibody comprises a heavy chain and a light chain, wherein the heavy chain comprises three CDR sequences comprising each of SEQ ID NOs: 22, 23, and 24, and the light chain comprises three CDR sequences comprising each of SEQ ID NOs: 25, 26, and 27.

In some embodiments, the anti-LIGHT antibody comprises a heavy chain comprising three CDR sequences comprising each of SEQ ID NOs: 28, 29, and 30. In some embodiments, the anti-LIGHT antibody comprises a light chain comprising three CDR sequences comprising each of SEQ ID NOs: 31, 32, and 33. In some embodiments, the antibody comprises a heavy chain and a light chain, wherein the heavy chain comprises three CDR sequences comprising each of SEQ ID NOs: 28, 29, and 30, and the light chain comprises three CDR sequences comprising each of SEQ ID NOs: 31, 32, and 33.

In some embodiments, the anti-LIGHT antibody comprises a heavy chain comprising three CDR sequences comprising each of SEQ ID NOs: 34, 35, and 36. In some embodiments, the anti-LIGHT antibody comprises a light chain comprising three CDR sequences comprising each of SEQ ID NOs: 37, 38, and 39. In some embodiments, the antibody comprises a heavy chain and a light chain, wherein the heavy chain comprises three CDR sequences comprising each of SEQ ID NOs: 34, 35, and 36, and the light chain comprises three CDR sequences comprising each of SEQ ID NOs: 37, 38, and 39.

In some embodiments, the anti-LIGHT antibody comprises a heavy chain comprising three CDR sequences comprising each of SEQ ID NOs: 40, 41, and 42. In some embodiments, the anti-LIGHT antibody comprises a light chain comprising three CDR sequences comprising each of SEQ ID NOs: 43, 44, and 45. In some embodiments, the antibody comprises a heavy chain and a light chain, wherein the heavy chain comprises three CDR sequences comprising each of SEQ ID NOs: 40, 41, and 42, and the light chain comprises three CDR sequences comprising each of SEQ ID NOs: 43, 44, and 45.

In some embodiments, the anti-LIGHT antibody comprises a heavy chain comprising three CDR sequences comprising each of SEQ ID NOs: 46, 47, and 48. In some embodiments, the anti-LIGHT antibody comprises a light chain comprising three CDR sequences comprising each of SEQ ID NOs: 49, 50, and 51. In some embodiments, the antibody comprises a heavy chain and a light chain, wherein the heavy chain comprises three CDR sequences comprising each of SEQ ID NOs: 46, 47, and 48, and the light chain comprises three CDR sequences comprises each of SEQ ID NOs: 49, 50, and 51.

In some embodiments, the anti-LIGHT antibody may comprise the CDR sequences of the antibodies which are described in US2013/0323240 and U.S. Pat. No. 8,524,869 B2, which are incorporated herein by reference. For example, in some embodiments, the anti-LIGHT antibody comprises a heavy chain comprising three CDR sequences comprising each of SEQ ID NOs: 52, 53, and 54, respectively. In some embodiments, the anti-LIGHT antibody comprises a light chain comprising three CDR sequences comprising each of SEQ ID NOs: 55, 56, and 57, respectively. In some embodiments, the antibody comprises a heavy chain and a light chain, the heavy chain comprising three CDR sequences comprising each of SEQ ID NOs: 52, 53, and 54, respectively, and the light chain comprising three CDR sequences comprising each of SEQ ID NOs: 55, 56, and 57, respectively.

In some embodiments, the anti-LIGHT antibody comprises a heavy chain variable region sequence comprising SEQ ID NO:58 or that is at least 85%, at least 90%, at least 95%, at least 98%, or at least 99% identical to SEQ ID NO:58. In some embodiments, the anti-LIGHT antibody comprises a light chain variable region sequence comprising SEQ ID NO:59 or that is at least 85%, at least 90%, at least 95%, at least 98%, or at least 99% identical to SEQ ID NO:59. In some embodiments, the anti-LIGHT antibody comprises a heavy chain comprising SEQ ID NO:58 or that is at least 85%, at least 90%, at least 95%, at least 98%, or at least 99% identical to SEQ ID NO:58. In some embodiments, the anti-LIGHT antibody comprises a light chain comprising SEQ ID NO:59 or that is at least 85%, at least 90%, at least 95%, at least 98%, or at least 99% identical to SEQ ID NO:59. In some embodiments, the anti-LIGHT antibody comprises both a heavy chain comprising SEQ ID NO:58 or that is at least 85%, at least 90%, at least 95%, at least 98%, or at least 99% identical to SEQ ID NO:58 and a light chain comprising SEQ ID NO:59 or that is at least 85%, at least 90%, at least 95%, at least 98%, or at least 99% identical to SEQ ID NO:59.

In some embodiments, the anti-LIGHT antibody may comprise a heavy chain and a light chain together comprising one of the following sets of CDR-H1, CDR-H2, CDR-H3, CDR-L1, CDR-L2, and CDR-L3 sequences described in the sequence listing from
US2013/0323240: SEQ ID NOs: 18, 19, 20 and SEQ ID NOs: 38, 41, 42 of
US2013/0323240; SEQ ID NOs: 18, 19, 21 and SEQ ID NOs: 39, 41, 42 of
US2013/0323240; SEQ ID NOs: 18, 19, 22 and SEQ ID NOs: 40, 41, 42 of
US2013/0323240; SEQ ID NOs: 23, 24, 25 and SEQ ID NOs: 43, 44, 45 of
US2013/0323240; SEQ ID NOs: 26, 27, 28 and SEQ ID NOs: 46, 47, 48 of
US2013/0323240; SEQ ID NOs: 29, 30, 31 and SEQ ID NOs: 49, 50, 51 of
US2013/0323240; SEQ ID NOs: 32, 33, 34 and SEQ ID NOs: 52, 53, 54 of
US2013/0323240; and SEQ ID NOs: 35, 36, 37 and SEQ ID NOs: 55, 50, 51 of
US2013/0323240.

In some embodiments, the anti-LIGHT antibody comprises the CDR sequences of the 18E04, 98C07, 1CO2, 1C06, 13H04, 31A10, 98C07, 42A02, 29CO2, 14B09, 117C06, 114F05, and 62C01 antibodies described in WO 2015/107331, which is also incorporated by reference herein.

For example, in some embodiments, the anti-LIGHT antibody comprises a heavy chain comprising three CDR sequences comprising each of SEQ ID NOs: 60, 61, and 62, respectively. In some embodiments, the anti-LIGHT antibody comprises a light chain comprising three CDR sequences comprising each of SEQ ID NOs: 63, 64, and 65, respectively. In some embodiments, the antibody comprises a heavy chain and a light chain, the heavy chain comprising three CDR sequences comprising each of SEQ ID NOs: 60, 61, and 62, respectively, and the light chain comprising three CDR sequences comprising each of SEQ ID NOs: 63, 64, and 65, respectively.

In some embodiments, the anti-LIGHT antibody comprises a heavy chain comprising three CDR sequences comprising each of SEQ ID NOs: 66, 67, and 68, respectively. In some embodiments, the anti-LIGHT antibody comprises a light chain comprising three CDR sequences comprising each of SEQ ID NOs: 69, 70, and 71, respectively. In some embodiments, the antibody comprises a heavy chain and a light chain, the heavy chain comprising three CDR sequences comprising each of SEQ ID NOs: 66, 67, and 68, respectively, and the light chain comprising three CDR sequences comprising each of SEQ ID NOs: 69, 70, and 71, respectively.

In some embodiments, the anti-LIGHT antibody comprises a heavy chain comprising three CDR sequences comprising each of SEQ ID NOs: 72, 73, and 74, respectively. In some embodiments, the anti-LIGHT antibody comprises a light chain comprising three CDR sequences comprising each of SEQ ID NOs: 75, 76, and 77, respectively. In some embodiments, the antibody comprises a heavy chain and a light chain, the heavy chain comprising three CDR sequences comprising each of SEQ ID NOs: 72, 73, and 74, respectively, and the light chain comprising three CDR sequences comprising each of SEQ ID NOs: 75, 76, and 77, respectively.

In some embodiments, the anti-LIGHT antibody comprises a heavy chain comprising three CDR sequences comprising each of SEQ ID NOs: 78, 79, and 80, respectively. In some embodiments, the anti-LIGHT antibody comprises a light chain comprising three CDR sequences comprising each of SEQ ID NOs: 81, 82, and 83, respectively. In some embodiments, the antibody comprises a heavy chain and a light chain, the heavy chain comprising three CDR sequences comprising each of SEQ ID NOs: 78, 79, and 80, respectively, and the light chain comprising three CDR sequences comprising each of SEQ ID NOs: 81, 82, and 83, respectively.

### Free LIGHT Detection Assays

Most currently available assays only measure total LIGHT, which includes LIGHT bound to its receptors, including DcR3. Total LIGHT may not provide as accurate of a picture of the levels of LIGHT causing disease, which may be free, unbound LIGHT. Thus, it may be desirable to use an assay that measures free LIGHT alone when the methods include detection of free LIGHT.

### Example 1

There is an existing clinical study protocol for an escalating dose, open-label, signal-finding study to evaluate the safety, tolerability, and short-term efficacy of an anti-LIGHT monoclonal antibody in adults with moderate to severe active Crohn's Disease who have failed prior treatment with an anti-TNFα agent, with and without loss of function mutations in decoy receptor 3 (DcR3). The anti-LIGHT monoclonal antibody comprises the following six CDRs: a heavy chain CDR having an amino acid sequence of SEQ ID NO: 2; a heavy chain CDR having an amino acid sequence of SEQ ID NO: 3; a heavy chain CDR having an amino acid sequence of SEQ ID NO: 4; a light chain CDR having an amino acid sequence of SEQ ID NO: 5; a light chain CDR having an amino acid sequence of SEQ ID NO: 6; and a light chain CDR having an amino acid sequence of SEQ ID NO: 7. In some embodiments the anti-LIGHT monoclonal antibody has a variable heavy chain (VH) having an amino acid sequence of SEQ ID NO: 84 and a variable light chain (VL) having an amino acid sequence of SEQ ID NO: 85. In some embodiments the anti-LIGHT monoclonal antibody has a heavy chain having an amino acid sequence of SEQ ID NO: 8 and a light chain having an amino acid sequence of SEQ ID NO: 9. The protocol is being amended to include subjects with ulcerative colitis who have failed prior treatment with an anti-TNFα agent. Below are portions of the existing Crohn's Disease protocol, which are now applicable to subjects being added to the study having UC.

Study Objectives and Endpoints

The primary objective of this study is to evaluate the safety and tolerability of the anti-LIGHT monoclonal antibody administered by SQ injection to adults with moderate to severe, active CD who have failed prior treatment with an anti-tumor necrosis factor alpha (anti-TNFα) agent.

The secondary objectives of this study are to: estimate plasma concentrations of the anti-LIGHT monoclonal antibody administered by SQ injection to adults with moderate to severe, active CD; and to evaluate response to treatment with the anti-LIGHT monoclonal antibody administered by SQ injection to adults with moderate to severe, active CD.

### Study Design

This is a Phase 1b, multi-center, open-label, dose-escalation, signal-finding study to evaluate the safety, tolerability, PK and short-term efficacy of the anti-LIGHT monoclonal antibody in adults with moderate to severe, active CD who have previously failed anti-TNFα treatment.

Four subjects with Crohn's Disease who satisfy all eligibility criteria are enrolled in each of 2 dose cohorts. The first cohort receives the anti-LIGHT monoclonal antibody 1.0 mg/kg SQ every 14 (q14) days.

Dose escalation proceeds after completion of the first cohort based on review of cumulative safety, tolerability, pharmacokinetic, and efficacy data by Data Monitoring Committee and after a decision is made to progress to the second cohort. The estimated dose escalation for the second cohort is 3.0 mg/kg SQ q14 days, if permitted by safety data review.

Each subject's participation includes a screening period, which if required includes a 12-week wash-out period for subjects receiving biologic treatment or who have received biologic treatment within 12 weeks of the Screening Visit. For subjects requiring wash-out, there is optionally a 1—to 14-day time period between the screening visit and the start of the wash-out period, as necessary. With the exception of subjects requiring wash-out of the biologic certolizumab pegol (Cimzia), only those subjects without detectable biologic levels after 8 weeks of wash-out are allowed to enter the study after confirmation of undetectable levels; all other subjects (including those receiving certolizumab pegol [Cimzia]) are required to complete a full 12-week wash-out period. The wash-out period includes the time period from the last dose received prior to the Screening Visit. Subjects not requiring a biologic wash-out period are allowed to enter the study after review and confirmation of eligibility at screening. Screening is followed by an 8-week, open-label treatment period, and a safety follow-up visit approximately 4 weeks after the last dose. The maximum study duration is 26 weeks.

Study visits occur at screening and on Days 0, 7, 14, 21, 28, 35, 42, 49 and 56. The safety follow-up visit occurs on Day 84.

Study Periods

The study includes a screening period, an open-label treatment period, and a safety follow-up visit. The 1.0 mg/kg dose cohort completes the study periods described below. Following a review of the safety data, a decision is made.

### Number of Subjects

Four subjects are enrolled in each of the 2 planned dose cohorts for a maximum of 8 study subjects. Subjects who withdraw from the study prematurely prior to a third dose are permitted to be replaced.

### Treatment Assignment

The first cohort of subjects are assigned to the 1.0 mg/kg dose of the anti-LIGHT monoclonal antibody. Subjects are assigned to the second dose cohort, after the DMC review of the safety data from the first cohort, and provided study stopping criteria are not met.

### Subject Inclusion Criteria

Subjects that meet all of the following inclusion criteria are eligible for enrollment in the study:
1. Subject is able to speak English fluently and provided written informed consent for this study.
2. Subject is male or female, >18 to 75 years of age.
3. Subject has a documented diagnosis of CD via endoscopy/colonoscopy and histological confirmation.
4. Subject has moderate to severe, active CD as evidenced by Simple Endoscopy Score for Crohn's Disease (SES-CD) score of and histological confirmation.
5. Subject has failed treatment with an approved therapeutic dose of an anti-TNFα monoclonal antibody treatment with either no initial response (primary non-responder) or an initial response to induction with subsequent lost response (secondary non-responder) as defined below.
6. Subject is permitted to receive concurrent treatment with an oral corticosteroid, and/orazathioprine or 6-mercaptopurine (6-MP) or methotrexate (MTX).
7. Subject agrees to be genotyped at the DcR3 locus.

A primary non-responder is defined as a subject for whom treatment with infliximab, adalimumab, or certolizumab pegol produced an inadequate initial response. Inadequate initial response symptom details occur≥2 weeks after the last dose of induction therapy. The algorithm for defining inadequate initial response is shown below. Subjects categorized as primary non-responders meet both parts of the algorithm. Documentation required includes dates and doses of failed induction therapy and lack of response details around disease activity recorded by a treating clinician.

The following algorithm is used for inadequate initial response to current or prior therapy with infliximab, adalimumab, or certolizumab pegol. The subject has received induction doses of either infliximab (2 or 3 doses of >5 mg/kg), adalimumab (dose of 160 mg followed by a dose of >80 mg or, dose of 80 mg followed by a dose of >40 mg), or certolizumab pegol (2 or 3 doses of >400 mg); and the subject did not initially respond to these induction doses as documented by the presence of at least 1 of the following signs or symptoms related to Crohn's Disease activity: lack of improvement or worsening in stool frequency; lack of improvement or worsening in daily abdominal pain; occurrence, lack of improvement, or worsening of fever associated with Crohn's Disease; recurring drainage from a previously non-draining fistula or development of a new draining fistula; lack of improvement or worsening in rectal bleeding; or initiation or increase in antidiarrheal medication.

A secondary non-responder is defined as a subject for whom treatment with infliximab, adalimumab, or certolizumab pegol produced an initial response followed by a loss of response. Loss of response details occurs>2 weeks after last dose of maintenance therapy. The algorithm for defining loss of response is described below. Subjects categorized as secondary non-responders meet both parts of the algorithm. Documentation required includes dates and doses of induction and maintenance, initial response and subsequent loss of response including details around disease activity recorded by a treating clinician. The following is the algorithm for loss of response to prior therapy with infliximab, adalimumab, or certolizumab pegol. The subject responded to induction therapy at doses described above and received at least 2 maintenance doses of: infliximab (>5 mg/kg), adalimumab (dose>40 mg or, if failed as a pediatric dose of >20 mg), or certolizumab pegol (>400 mg); and the subject did not respond to these maintenance doses as documented by the presence of at least 1 of the following signs or symptoms related to Crohn's Disease activity: worsening in stool frequency; worsening in daily abdominal pain; occurrence, or worsening of fever associated with Crohn's Disease; recurring drainage from a previously non-draining fistula or development of a new draining fistula; worsening in rectal bleeding; or initiation or increase in antidiarrheal medication.

### Subject Exclusion Criteria

Subjects who meet any of the following exclusion criteria are not eligible for enrollment in the study:
1. Subject has a diagnosis of ulcerative colitis (UC) or indeterminate colitis.
2. Subject is unable to tolerate or unwilling to undergo study procedures including endoscopy and biopsy during the study.
3. Subject has signs or symptoms of bowel obstruction with small bowel imaging supporting obstruction.
4. Subject has short bowel syndrome as determined by the investigator.
5. Subject has a current functional colostomy or ileostomy.
6. Subject had a surgical bowel resection within the past 6 months prior to screening or is planning any resection during the study period.
7. Clinical suspicion of intra-abdominal abscesses exist, in the opinion of the investigator.
8. Subject has concurrent bowel dysplasia or a history of bowel dysplasia in the 5 years prior to screening.
9. Subject has a known, active and/or positive test for C. difficile infection.
10. Subject has history of or current diagnosis of any cancer excluding cancers that have been cured by surgical excision (e.g., non melanoma skin cancers).
11. Subject has a history of a lymphoproliferative disorder, including lymphoma, or signs and symptoms suggestive of lymphoproliferative disease at any time.
12. Subject has history of or active TB infection or positive TB testing at screening.
13. Subject has known concurrent viral hepatitis, or acquired immune deficiency syndrome (AIDS) or known human immunodeficiency virus (HIV) infection.
14. Subject has been treated with natalizumab (TYSABRI®).
15. Subject has not completed his/her primary vaccination series (particularly hepatitis B, varicella, measles/mumps/rubella) unless immunity documented with blood titers.
16. Subject received any live attenuated vaccine, such as varicella-zoster, oral polio, orrubella, within 3 months prior to the baseline visit.
17. Subject has any of the following abnormal screening laboratory test results: clinically significant ECG abnormalities; aspartate transaminase (AST), alanine transaminase (ALT) or total bilirubin>ULN; hemoglobin<10 g/dL; absolute neutrophil count<1500 cell/mm³, or; estimated glomerular filtration rate<60 mL/min/1.73 m².
18. Subject has abnormal vital signs during Screening (Visit 1) or prior to enrollment at the baseline visit (Visit 2).
19. Subject is pregnant or a nursing mother.
20. Subject is sexually active and not on effective contraception.
21. Subject has a history of drug abuse that may inhibit participation in the clinical study.
22. Subject has a current or recent history (within 6 months prior to screening) of significant and severe renal, hepatic, hematological, gastrointestinal (other than CD or conditions outlined above), endocrine, pulmonary, cardiac, or neurological disease.
23. Subject has any other clinically significant mental or physical illness or infection that, inthe opinion of the investigator, might confound the results of the study, pose additional risk to the subject by their participation, or prevent or impede the subject from completing the study.
24. There is any concern on the part of the investigator regarding the subject's safety, compliance, or suitability with respect to his/her participation in the study.

### Treatment of Subjects

Subjects in each dose cohort receive the investigational product as a single SQ injection in the abdomen in a zone of 4 to 10 cm from the umbilicus with the injection site rotated with each subsequent dose. The dose of the anti-LIGHT monoclonal antibody is administered on Days 0, 14, 28, and 42. After Day 0, injections occur within ±3 days of the scheduled 14-day intervals.

Subjects in the first dose cohort receive the anti-LIGHT monoclonal antibody 1.0 mg/kg for the entire 8-week, open-label treatment period. Data from this cohort are reviewed, after all subjects have completed the treatment period and its associated assessments and before the second cohort is enrolled. It is anticipated that there are a minimum of 2 weeks between subject last visit and the review of cumulative safety, tolerability, pharmacokinetic and efficacy data.

Eligible subjects in the second dose cohort receive the anti-LIGHT monoclonal antibody 3.0 mg/kg for the entire 8-week, open-label treatment period. Data from the second cohort are reviewed, after all subjects have completed the treatment period and its associated assessments.

### Permitted Therapies

Subjects are permitted to receive concurrent treatment with an oral corticosteroid, and/or azathioprine, 6-MP or MTX. Subjects are not allowed to have their dose of these medications increased during the study. If a dose increase of a concomitant permitted therapy is required, the subject is discontinued from the study utilizing the Early Termination visit procedures. Concurrent treatment with an oral corticosteroid, and/or azathioprine, 6-MP or MTX is defined as follows: Oral corticosteroid—Prednisone dose not exceeding 40 mg/day, with a stable dose for at least 2 weeks prior to baseline; Azathioprine or 6-MP— Azathioprine dose of at least 2 mg/kg/day or 6-MP dose of 1 to 1.5 mg/kg/day rounded to the nearest available tablet formulation, or a dose that is the highest tolerated for the subject, in the opinion of the investigator, for at least 8 weeks prior to baseline with a stable dose for at least 4 weeks prior to baseline; or MTX dose of 25 mg/week during study, either SQ, intramuscularly, or orally, for at least 8 weeks prior to baseline with a stable dose for at least 4 weeks prior to baseline.

Doses of these therapies are permitted to be decreased during the study; however, all doses are within the combinations and dose ranges specified above. These changes are recorded on the concomitant medication eCRF.

For subjects on corticosteroids at the time of study enrollment, weaning during the study is done according to the following rules: corticosteroid dose of >20 mg and a maximum taper rate per week of 10 mg; 10 to <20 mg and a maximum taper rate per week of 5 mg; or <10 mg and a maximum taper rate per week of 2.5 mg. If a deviation from the permitted concomitant therapy combinations, dose ranges or weaning schedule are necessary, the subject is withdrawn from study participation.

### Prohibited Therapies

Concomitant use of biologic treatments during the study is prohibited including use of anakinra (KINERET®, Amgen), abatacept (ORENCIA®, Bristol-Myers Squibb), or tocilizumab (ACTEMRA®, Genentech). A wash-out period of up to 12 weeks is required prior to study enrollment (Visit 2) and after confirmation of eligibility from screening procedures performed at Visit 1 for all biologic treatments received within 12 weeks of the Screening Visit. Prior treatment with natalizumab (TYSABRI®, Biogen) excludes subjects from participation.

Vaccination with live or attenuated virus 3 months prior to screening and at any time during the study is prohibited.

### Randomization and Blinding

All subjects receive the anti-LIGHT monoclonal antibody in an open-label manner.

### Investigational Product

The anti-LIGHT monoclonal antibody is the investigational product used in this study having a heavy chain sequence of SEQ ID NO: 8, and a light chain sequence of SEQ ID NO: 9. It is administered in the dosage form 150 mg/mL solution. The unit dose is 1.0 mg/kg or 3.0 mg/kg. The route of administration is SQ injection in the abdomen in a zone of 4 to 10 cm from the umbilicus with the injection site rotated with each subsequent dose. It is in a colorless to slightly yellowish brown solution. It is manufactured by sanofi-aventis group.

### Administration

The anti-LIGHT monoclonal antibody is administered by SQ injection in the abdomen in a zone of 4 to 10 cm from the umbilicus with the injection site rotated with each subsequent dose.

### Study Duration

The overall study duration is approximately 26 weeks (including up to 14 weeks of screening [inclusive of wash-out of up to 12 weeks, if required], 56 days of open-label treatment, and a follow-up visit approximately 28 days after the final dose of investigational product).

The planned sequence and maximum duration of the study periods is as follows:
1. Screening period: approximately 14 weeks
2. Wash-out period, if applicable: Up to 12 weeks from the subject's last dose of biological treatment
3. Open-label treatment period: 56 days (beginning on Day 0 with doses administered q14 days)
4. Follow-up: 28 days after the last dose of investigational product. The maximum study duration for each subject is approximately 26 weeks.

The maximum treatment duration for each subject is approximately 56 days, with SQ injections beginning on Day 0 and continuing every 14 (±3) days through Day 42.

Note: Extensions of the screening window to accommodate clinical laboratory re-testing timeframes (excluding endoscopy with biopsy) are permitted but are not allowed to exceed a total screening time of 16 weeks.

### Safety Assessments

Safety assessments include monitoring of AEs, clinical laboratory tests, vital signs measurements, physical examinations (including measurement of weight) and 12-lead ECG parameters. Demographic information and medical and medication histories are obtained at the screening visit.

All safety assessments are recorded on the appropriate eCRF.

### Genotyping for Decoy Receptor 3

Two milliliters of saliva for genotyping are collected in a designated collection vehicle according to the manufacturer's instructions.

Deoxyribonucleic acid (DNA) is isolated from the saliva samples from each study subject and then evaluated for genetic alteration in TNFRSF6B encoding for the protein DcR3 or alterations in at least one DcR3 network gene. All genotyping is performed in a CLIA certified laboratory specified in the laboratory manual(s) and guidance(s). Any remaining DNA samples are stored for future biomarker studies.

### LIGHT, Cytokines, RNA Sequencing and Flow Cytometry Exploratory Analyses

Blood samples are collected for exploratory analyses. Exploratory analyses include but are not limited to LIGHT biomarker, cytokines (e.g. IL-1 beta, IL-6, IL-8, TNF-alpha, and other exploratory cytokines), ribonucleic acid (RNA) sequencing; flow cytometry of peripheral blood leukocytes.

Endoscopy with Biology and Histology

All subjects who enroll in the study undergo an endoscopy with biopsy at screening and again at Day 56 (Visit 10) or at early termination. Screening endoscopies are optionally performed either as a stand-alone endoscopy for purposes of this protocol or as a clinically-required endoscopy, provided consenting procedures for this study are completed prior to endoscopy.

Endoscopy evaluation uses the SES-CD. The SES-CD is a simple, easy-to-use endoscopic scoring system developed specifically for CD. It assesses 4 variables: size of ulcers, percentage of ulcerated surface, percentage of affected surface and the presence of narrowing across 4 categories per variable on a scale of 0 to 3 (Daperno M, D′Haens G, Van Assche G et al. Development and validation of a new, simplified endoscopic activity score for Crohn's disease: the SES-CD. Gastroinest Endosc. 2004 October; 60(4):505-12). Biopsies taken at screening are assessed for histological confirmation of disease. Each subject has screening and Visit 10/ET biopsy samples retained for evaluation of exploratory parameters (which may include but is not limited to DcR3, LIGHT, HVEM and LTβR) which could optionally occur after study completion.

### Patient-reported Assessment of Well-being, Abdominal Pain and Stool Frequency

All subjects who enroll in the study report their daily assessment of well-being, abdominal pain and stool frequency including loose and/or watery stools via a diary (electronic or hard copy). Abdominal pain is assessed on a scale of 0 to 3 with higher values indicating greater pain severity. The stool frequency including number of loose and/or watery stools per day, equivalent to a score of a 6 or 7 on the Bristol Stool Scale, is recorded.

Loose stools are described as fluffy pieces with ragged edges, a mushy stool. Watery stools are described as watery, no solid pieces (O'Donnell et al., Detection of pseudodiarrhoea by simple clinical assessment of intestinal transit rate, Br. Med. 1 1990; 300:439-40).

### Quality of Life Assessment—Inflammatory Bowel Disease Questionnaire

The IBD-Q is a 32 item questionnaire validated to measure quality of life in Crohn's Disease. The IBD-Q assesses the dimensions of bowel function, emotional status, systemic symptoms and social function (Guyatt et al., A new measure of health status for clinical trials in inflammatory bowel disease, Gastroenterol., 1989:96;804-10). The IBD-Q is completed by all subjects at screening (Visit 1), before dosing (Visit 2), and at the end of the open-label treatment period or early termination (Visit 10/ET).

The following Table 1 provides the sequences referred to in this application.

TABLE 1
Table of Sequences

| SEQ | | |
|---|---|---|
| ID | | |
| NO | DESCRIPTION | SEQUENCE |
| 1 | Human DcR3 | MRALEGPGLS LLCLVLALPA LLPVPAVRGV |
| | amino acid | AETPTYPWRD |
| | sequence | AETGERLVCA QCPPGTFVQR PCRRDSPTTC |
| | | GPCPPRHYTQ FWNYLERCRY CNVLCGEREE |
| | | EARACHATHN RACRCRTGFF AHAGFCLEHA |
| | | SCPPGAGVIA PGTPSQNTQC QPCPPGTFSA |
| | | SSSSSEQCQP HRNCTALGLA LNVPGSSSHD |
| | | TLCTSCTGFP LSTR VPGAEE CERAVIDFVA |
| | | FQDISIKRLQ RLLQALEAPE GWGPTPRAGR |
| | | AALQLKLRRR LTELLGAQDG ALLVRLLQAL |
| | | RVARMPGLER SVRERFLPVH |
| 2 | Heavy Chain | GYNWH |
| | (HC) CDR1 | |
| | antibody F19 | |
| 3 | HC CDR2 | EITHSGSTNYNPSLKS |
| | antibody F19 | |
| 4 | HC CDR3 | EIAVAGTGYYGMDV |
| | antibody F19 | |
| 5 | LC CDR1 | RASQGINSAFA |
| | antibody F19 | |
| 6 | LC CDR2 | DASSLES |
| | antibody F19 | |
| 7 | LC CDR3 | QQFNSYPLT |
| | antibody F19 | |
| 8 | Heavy chain | QVQLQQWGAG LLKPSETLSL TCAVYGGSFS |
| | antibody F19 | GYNWHWIRQP PGKGLEWIGE ITHSGSTNYN |
| | | PSLKSRVTIS VDTSKNQFSL KLSSVTAADT |
| | | AVYYCVREIA VAGTGYYGMD VWGQGTTVTV |
| | | SSASTKGPSV FPLAPCSRST SESTAALGCL |
| | | VKDYFPEPVT VSWNSGALTS GVHTFPAVLQ |
| | | SSGLYSLSSV VTVPSSSLGT KTYTCNVDHK |
| | | PSNTKVDKRV ESKYGPPCPP CPAPEFEGGP |
| | | SVFLFPPKPK DTLMISRTPE VTCVVVDVSQ |
| | | EDPEVQFNWY VDGVEVHNAK TKPREEQFNS |
| | | TYRVVSVLTV LHQDWLNGKE YKCKVSNKGL |
| | | PSSIEKTISK AKGQPREPQV YTLPPSQEEM |
| | | TKNQVSLTCL VKGFYPSDIA VEWESNGQPE |
| | | NNYKTTPPVL DSDGSFFLYS RLTVDKSRWQ |
| | | EGNVFSCSVM HEALHNHYTQ KSLSLSLG |
| 9 | Light chain | AIQLTQSPSS LSASVGDRVT ITCRASQGIN |
| | antibody F19 | SAFAWYQQKP GKAPKLLIYD ASSLESGVPS |
| | | RFSGSGSGTD FTLTISSLQP EDFATYYCQQ |
| | | FNSYPLTFGG GTKVEIKRTV AAPSVFIFPP |
| | | SDEQLKSGTA SVVCLLNNFY PREAKVQWKV |
| | | DNALQSGNSQ ESVTEQDSKD STYSLSSTLT |
| | | LSKADYEKHK VYACEVTHQG LSSPVTKSEN RGEC |
| 10 | Alternative LC | RASRGINSAFA |
| | CDR1 antibody | |
| | F19 | |
| 11 | Alternative LC | DASSLES |
| | CDR2 antibody | |
| | F19 | |
| 12 | Alternative LC | QQFNSYPLT |
| | CDR3 antibody | |
| | F19 | |
| 13 | Alternative LC | RVSQGISSYLN |
| | CDR1 antibody | |
| | F19 | |
| 14 | Alternative LC | SASNLQS |
| | CDR2 antibody | |
| | F19 | |
| 15 | Alternative LC | ARTNAPPT |
| | CDR3 antibody | |
| | F19 | |
| 16 | Alternative LC | RMSQGISSYLA |
| | CDR1 antibody | |
| | F19 | |
| 17 | Alternative LC | AASTLQS |
| | CDR2 antibody | |
| | F19 | |
| 18 | Alternative LC | QQYYSFPYT |
| | CDR3 antibody | |
| | F19 | |
| 19 | Alternative LC | RASQGVSSYLA |
| | CDR1 antibody | |
| | F19 | |
| 20 | Alternative LC | DASNRAT |
| | CDR2 antibody | |
| | F19 | |
| 21 | Alternative LC | QQRSNWHP |
| | CDR3 antibody | |
| | F19 | |
| 22 | HC CDR1 | RFNMN |
| | antibody E1 | |
| 23 | HC CDR2 | YISSSSYTIYYADSVKG |
| | antibody E1 | |
| 24 | HC CDR3 | SIAAFDY |
| | antibody E1 | |
| 25 | LC CDR1 | RASQGISSALA |
| | antibody E1 | |
| 26 | LC CDR2 | DASSLES |
| | antibody E1 | |
| 27 | LC CDR3 | QQFNSYRT |
| | antibody E1 | |
| 28 | Alternative LC | RASQSVSSSYLT |
| | CDR1 antibody | |
| | E1 | |
| 29 | Alternative LC | GASSRAT |
| | CDR2 antibody | |
| | E1 | |
| 30 | Alternative LC | QQYGSSMYT |
| | CDR3 antibody | |
| | E1 | |
| 31 | Alternative LC | RASQSVSSSYLA |
| | CDR1 antibody | |
| | E1 | |
| 32 | Alternative LC | GASNRAT |
| | CDR2 antibody | |
| | E1 | |
| 33 | Alternative LC | QQYGSSPWT |
| | CDR3 antibody | |
| | E1 | |
| 34 | HC CDR1 | NAWMS |
| | antibody E13 | |
| 35 | HC CDR2 | RIKSKIDGGTTDYAAPVKG |
| | antibody E13 | |
| 36 | HC CDR3 | AMAGAFGF |
| | antibody E13 | |
| 37 | LC CDR1 | RASQSVSSSYLA |
| | antibody E13 | |
| 38 | LC CDR2 | GASSRAT |
| | antibody E13 | |
| 39 | LC CDR3 | QQYGSSPMYT |
| | antibody E13 | |
| 40 | HC CDR1 | SGGYYWS |
| | antibody E63 | |
| 41 | HC CDR2 | YIYYSGSTNYNPSLKS |
| | antibody E63 | |
| 42 | HC CDR3 | WITMFRGVGFDP |
| | antibody E63 | |
| 43 | LC CDR1 | RASQSIGSSLH |
| | antibody E63 | |
| 44 | LC CDR2 | YASQSFS |
| | antibody E63 | |
| 45 | LC CDR3 | RQSSSLPLT |
| | antibody E63 | |
| 46 | HC CDR1 | GYYWN |
| | antibody F23 | |
| 47 | HC CDR2 | EINQYNPSLKS |
| | antibody F23 | |
| 48 | HC CDR3 | EIAIADKGYYGLDV |
| | antibody F23 | |
| 49 | LC CDR1 | RASQGISSALA |
| | antibody F23 | |
| 50 | LC CDR2 | DASSLES |
| | antibody F23 | |
| 51 | LC CDR3 | QQFNSYPLT |
| | antibody F23 | |
| 52 | HC CDR1 | SYYIH |
| 53 | HC CDR2 | PGSDITKYNEKFKG |
| 54 | HC CDR3 | GISTYSAMDF |
| 55 | LC CDR1 | KASQDVGTAVA |
| 56 | LC CDR2 | WASTRHT |
| 57 | LC CDR3 | QQYSSYPLT |
| 58 | HC variable | QVQLVQSGAE VKKPGASVKV SCKASGYTFT |
| | region | SYYIHWVRQA PGQRLEWMGW IFPGSDITKY |
| | | NEKFKGRVTI TRDTSASTAY MELSSLRSED |
| | | TAVYYCARED YGISTYSAMD FWGQGTLVTV SS |
| 59 | LC variable | DIQLTQSPSF LSASVGDRVT ITCKASQDVG |
| | region | TAVAWYQQKP GKAPKLLIYW ASTRHTGVPS |
| | | RFSGSGSGTE FTLTISSLQP EDFATYYCQQ |
| | | YSSYPLTFGQ GTKVEIKR |
| 60 | HC CDR1 of | HFDIN |
| | 18E04 | |
| 61 | HC CDR2 of | WMNPDSDNTDYAQEFQG |
| | 18E04 | |
| 62 | HC CDR3 of | GGTTLDY |
| | 18E04 | |
| 63 | LC CDR1 of | SGDALPKKYAY |
| | 18E04 | |
| 64 | LC CDR2 of | EDSKRPS |
| | 18E04 | |
| 65 | LC CDR3 of | YSTDSSDNHVI |
| | 18E04 | |
| 66 | HC CDR1 of | DYYMS |
| | 98C07 | |
| 67 | HC CDR2 of | YISRSSFIYYSESVKG |
| | 98C07 | |
| 68 | HC CDR3 of | WELSPFDY |
| | 98C07 | |
| 69 | LC CDR1 of | RASQGISNYLA |
| | 98C07 | |
| 70 | LC CDR2 of | AASSLQS |
| | 98C07 | |
| 71 | LC CDR3 of | QQYNTYPFT |
| | 98C07 | |
| 72 | HC CDR1 of | YYGIS |
| | 1C02 | |
| 73 | HC CDR2 of | WISANSGNTNYAQKFQG |
| | 1C02 | |
| 74 | HC CDR3 of | GGVAVLEY |
| | 1C02 | |
| 75 | LC CDR1 of | WASQGISSYLA |
| | 1C02 | |
| 76 | LC CDR2 of | VASTLQS |
| | 1C02 | |
| 77 | LC CDR3 of | QQLKIYPLT |
| | 1C02 | |
| 78 | HC CDR1 of | DYYMN |
| | 1C06 | |
| 79 | HC CDR2 of | DISSRDNTIYYADSVKG |
| | 1C06 | |
| 80 | HC CDR3 of | ARERGFGDYFGMDV |
| | 1C06 | |
| 81 | LC CDR1 of | RASQDISSALA |
| | 1C06 | |
| 82 | LC CDR2 of | DASSLES |
| | 1C06 | |
| 83 | LC CDR3 of | QQFNTYPLT |
| | 1C06 | |
| 84 | Heavy chain | QVQLQQWGAGLLKP SETLSLTCAVYGGSFSGYNWH |
| | variable region | WIRQPPGKGLEWIGEITHSGSTNYNPSLK |
| | (VH) of antibody | SRVTISVDTSKNQFSLKLSSVTAADTAVYYCVR |
| | F19 | EIAVAGTGYYGMDVWG QGTTVTVSS |
| 85 | Light chain | AIQLTQSPSSLSASVGDRVTITCR |
| | variable region | ASQGINSAFAWYQQKPGKAPKLLI |
| | (VL) of antibody | YDASSLESGVPSRFSG SGSGTDFTLTISS |
| | F19 | LQPEDFATYYCQQFNSYPLTFGGGTKVEIK |

What is claimed is:

1. A method of treating ulcerative colitis or an inflammatory condition associated with ulcerative colitis, comprising administering an anti-LIGHT antibody to a subject in need thereof, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57.

2. A method of treating ulcerative colitis or an inflammatory condition associated with ulcerative colitis, comprising administering an anti-LIGHT antibody to a subject in need thereof, wherein the subject in need thereof has failed treatment with an approved therapeutic dose of an anti-TNFα monoclonal antibody with either no initial response or an initial response to induction with subsequent lost response, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57.

3. A method of treating ulcerative colitis or an inflammatory condition associated with ulcerative colitis, comprising administering an anti-LIGHT antibody to a subject in need thereof, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57;
wherein the anti-LIGHT antibody is administered at a dose of 1.0 mg/kg or 3.0 mg/kg every 14 days.

4. A method of treating ulcerative colitis or an inflammatory condition associated with ulcerative colitis, comprising administering an anti-LIGHT antibody to a subject in need thereof, wherein the subject in need thereof has failed treatment with an approved therapeutic dose of an anti-TNFα monoclonal antibody with either no initial response or an initial response to induction with subsequent lost response, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57;
wherein the anti-LIGHT antibody is administered at a dose of 1.0 mg/kg or 3.0 mg/kg every 14 days.

5. The method of any one of the preceding claims, wherein the anti-LIGHT antibody is administered subcutaneously.

6. The method of any one of the preceding claims, wherein the method further comprises assaying free LIGHT prior to, during, or after administration of the anti-LIGHT antibody.

7. The method of any one of the preceding claims, wherein the subject has elevated free LIGHT.

8. The method of any one of the preceding claims, wherein the subject is human.

9. The method of any one of the preceding claims, wherein the subject is an adult.

10. The method of any one of the preceding claims, wherein the subject is a pediatric subject.

11. The method of any one of the preceding claims, wherein the antibody comprises a variable heavy chain (VH) comprising an amino acid sequence of SEQ ID NO: 84.

12. The method of any one of the preceding claims, wherein the antibody comprises a variable light chain (VL) comprising an amino acid sequence of SEQ ID NO: 85.

13. The method of any one of the preceding claims, wherein the antibody comprises a heavy chain comprising an amino acid sequence of SEQ ID NO: 8.

14. The method of any one of the preceding claims, wherein the antibody comprises a light chain comprising an amino acid sequence of SEQ ID NO: 9.

15. The method of any one of claims 1-14, wherein administration of the anti-LIGHT antibody decreases the subject's endoscopic Mayo score.

16. The method of any one of claims 1-15, wherein administration of the anti-LIGHT antibody decreases the subject's total Mayo score.

17. The method of any one of claims 1-16, wherein administration of the anti-LIGHT antibody increases the subject's IBD-Q score.

18. The method of any one of the preceding claims, wherein administration of the anti-LIGHT antibody reduces serum free LIGHT in the subject.


SEQUENCE LISTING (85 sequences)

SEQ ID NO: 1 - protein, 300 aa, Homo sapiens
  REGION 1..300: note = misc_feature - Human DcR3 amino acid sequence
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 2 - protein, 5 aa, synthetic construct
  REGION 1..5: note = Synthetic: Heavy Chain (HC) CDR1 antibody F19
GYNWH

SEQ ID NO: 3 - protein, 16 aa, synthetic construct
  REGION 1..16: note = Synthetic: HC CDR2 antibody F19
EITHSGSTNYNPSLKS

SEQ ID NO: 4 - protein, 14 aa, synthetic construct
  REGION 1..14: note = Synthetic: HC CDR3 antibody F19
EIAVAGTGYYGMDV

SEQ ID NO: 5 - protein, 11 aa, synthetic construct
  REGION 1..11: note = Synthetic: LC CDR1 antibody F19
RASQGINSAFA

SEQ ID NO: 6 - protein, 7 aa, synthetic construct
  REGION 1..7: note = Synthetic: LC CDR2 antibody F19
DASSLES

SEQ ID NO: 7 - protein, 9 aa, synthetic construct
  REGION 1..9: note = Synthetic: LC CDR3 antibody F19
QQFNSYPLT

SEQ ID NO: 8 - protein, 448 aa, synthetic construct
  REGION 1..448: note = Synthetic: Heavy chain variable region antibody F19
QVQLQQWGAGLLKPSETLSLTCAVYGGSFSGYNWHWIRQPPGKGLEWIGEITHSGSTNYNPSLKSRVTISVDTSKNQFSLKLSSVTAADTAVYYCVREIAVAGTGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 214 aa, synthetic construct
  REGION 1..214: note = Synthetic: Light chain variable region antibody F19
AIQLTQSPSSLSASVGDRVTITCRASQGINSAFAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 11 aa, synthetic construct
  REGION 1..11: note = Synthetic: Alternative LC CDR1 antibody F19
RASRGINSAFA

SEQ ID NO: 11 - protein, 7 aa, synthetic construct
  REGION 1..7: note = Synthetic: Alternative LC CDR2 antibody F19
DASSLES

SEQ ID NO: 12 - protein, 9 aa, synthetic construct
  REGION 1..9: note = Synthetic: Alternative LC CDR3 antibody F19
QQFNSYPLT

SEQ ID NO: 13 - protein, 11 aa, synthetic construct
  REGION 1..11: note = Synthetic: Alternative LC CDR1 antibody F19
RVSQGISSYLN

SEQ ID NO: 14 - protein, 7 aa, synthetic construct
  REGION 1..7: note = Synthetic: Alternative LC CDR2 antibody F19
SASNLQS

SEQ ID NO: 15 - protein, 8 aa, synthetic construct
  REGION 1..8: note = Synthetic: Alternative LC CDR3 antibody F19
ARTNAPPT

SEQ ID NO: 16 - protein, 11 aa, synthetic construct
  REGION 1..11: note = Synthetic: Alternative LC CDR1 antibody F19
RMSQGISSYLA

SEQ ID NO: 17 - protein, 7 aa, synthetic construct
  REGION 1..7: note = Synthetic: Alternative LC CDR2 antibody F19
AASTLQS

SEQ ID NO: 18 - protein, 9 aa, synthetic construct
  REGION 1..9: note = Synthetic: Alternative LC CDR3 antibody F19
QQYYSFPYT

SEQ ID NO: 19 - protein, 11 aa, synthetic construct
  REGION 1..11: note = Synthetic: Alternative LC CDR1 antibody F19
RASQGVSSYLA

SEQ ID NO: 20 - protein, 7 aa, synthetic construct
  REGION 1..7: note = Synthetic: Alternative LC CDR2 antibody F19
DASNRAT

SEQ ID NO: 21 - protein, 8 aa, synthetic construct
  REGION 1..8: note = Synthetic: Alternative LC CDR3 antibody F19
QQRSNWHP

SEQ ID NO: 22 - protein, 5 aa, synthetic construct
  REGION 1..5: note = Synthetic: HC CDR1 antibody E1
RFNMN

SEQ ID NO: 23 - protein, 17 aa, synthetic construct
  REGION 1..17: note = Synthetic: HC CDR2 antibody E1
YISSSSYTIYYADSVKG

SEQ ID NO: 24 - protein, 7 aa, synthetic construct
  REGION 1..7: note = Synthetic: HC CDR3 antibody E1
SIAAFDY

SEQ ID NO: 25 - protein, 11 aa, synthetic construct
  REGION 1..11: note = Synthetic: LC CDR1 antibody E1
RASQGISSALA

SEQ ID NO: 26 - protein, 7 aa, synthetic construct
  REGION 1..7: note = Synthetic: LC CDR2 antibody E1
DASSLES

SEQ ID NO: 27 - protein, 8 aa, synthetic construct
  REGION 1..8: note = Synthetic: LC CDR3 antibody E1
QQFNSYRT

SEQ ID NO: 28 - protein, 12 aa, synthetic construct
  REGION 1..12: note = Synthetic: Alternative LC CDR1 antibody E1
RASQSVSSSYLT

SEQ ID NO: 29 - protein, 7 aa, synthetic construct
  REGION 1..7: note = Synthetic: Alternative LC CDR2 antibody E1
GASSRAT

SEQ ID NO: 30 - protein, 9 aa, synthetic construct
  REGION 1..9: note = Synthetic: Alternative LC CDR3 antibody E1
QQYGSSMYT

SEQ ID NO: 31 - protein, 12 aa, synthetic construct
  REGION 1..12: note = Synthetic: Alternative LC CDR1 antibody E1
RASQSVSSSYLA

SEQ ID NO: 32 - protein, 7 aa, synthetic construct
  REGION 1..7: note = Synthetic: Alternative LC CDR2 antibody E1
GASNRAT

SEQ ID NO: 33 - protein, 9 aa, synthetic construct
  REGION 1..9: note = Synthetic: Alternative LC CDR3 antibody E1
QQYGSSPWT

SEQ ID NO: 34 - protein, 5 aa, synthetic construct
  REGION 1..5: note = Synthetic: HC CDR1 antibody E13
NAWMS

SEQ ID NO: 35 - protein, 19 aa, synthetic construct
  REGION 1..19: note = Synthetic: HC CDR2 antibody E13
RIKSKIDGGTTDYAAPVKG

SEQ ID NO: 36 - protein, 8 aa, synthetic construct
  REGION 1..8: note = Synthetic: HC CDR3 antibody E13
AMAGAFGF

SEQ ID NO: 37 - protein, 12 aa, synthetic construct
  REGION 1..12: note = Synthetic: LC CDR1 antibody E13
RASQSVSSSYLA

SEQ ID NO: 38 - protein, 7 aa, synthetic construct
  REGION 1..7: note = Synthetic: LC CDR2 antibody E13
GASSRAT

SEQ ID NO: 39 - protein, 10 aa, synthetic construct
  REGION 1..10: note = Synthetic: LC CDR3 antibody E13
QQYGSSPMYT

SEQ ID NO: 40 - protein, 7 aa, synthetic construct
  REGION 1..7: note = Synthetic: HC CDR1 antibody E63
SGGYYWS

SEQ ID NO: 41 - protein, 16 aa, synthetic construct
  REGION 1..16: note = Synthetic: HC CDR2 antibody E63
YIYYSGSTNYNPSLKS

SEQ ID NO: 42 - protein, 12 aa, synthetic construct
  REGION 1..12: note = Synthetic: HC CDR3 antibody E63
WITMFRGVGFDP

SEQ ID NO: 43 - protein, 11 aa, synthetic construct
  REGION 1..11: note = Synthetic: LC CDR1 antibody E63
RASQSIGSSLH

SEQ ID NO: 44 - protein, 7 aa, synthetic construct
  REGION 1..7: note = Synthetic: LC CDR2 antibody E63
YASQSFS

SEQ ID NO: 45 - protein, 9 aa, synthetic construct
  REGION 1..9: note = Synthetic: LC CDR3 antibody E63
RQSSSLPLT

SEQ ID NO: 46 - protein, 5 aa, synthetic construct
  REGION 1..5: note = Synthetic: HC CDR1 antibody F23
GYYWN

SEQ ID NO: 47 - protein, 11 aa, synthetic construct
  REGION 1..11: note = Synthetic: HC CDR2 antibody F23
EINQYNPSLKS

SEQ ID NO: 48 - protein, 14 aa, synthetic construct
  REGION 1..14: note = Synthetic: HC CDR3 antibody F23
EIAIADKGYYGLDV

SEQ ID NO: 49 - protein, 11 aa, synthetic construct
  REGION 1..11: note = Synthetic: LC CDR1 antibody F23
RASQGISSALA

SEQ ID NO: 50 - protein, 7 aa, synthetic construct
  REGION 1..7: note = Synthetic: LC CDR2 antibody F23
DASSLES

SEQ ID NO: 51 - protein, 9 aa, synthetic construct
  REGION 1..9: note = Synthetic: LC CDR3 antibody F23
QQFNSYPLT

SEQ ID NO: 52 - protein, 5 aa, synthetic construct
  REGION 1..5: note = Synthetic: HC CDR1
SYYIH

SEQ ID NO: 53 - protein, 14 aa, synthetic construct
  REGION 1..14: note = Synthetic: HC CDR2
PGSDITKYNEKFKG

SEQ ID NO: 54 - protein, 10 aa, synthetic construct
  REGION 1..10: note = Synthetic: HC CDR3
GISTYSAMDF

SEQ ID NO: 55 - protein, 11 aa, synthetic construct
  REGION 1..11: note = Synthetic: LC CDR1
KASQDVGTAVA

SEQ ID NO: 56 - protein, 7 aa, synthetic construct
  REGION 1..7: note = Synthetic: LC CDR2
WASTRHT

SEQ ID NO: 57 - protein, 9 aa, synthetic construct
  REGION 1..9: note = Synthetic: LC CDR3
QQYSSYPLT

SEQ ID NO: 58 - protein, 122 aa, synthetic construct
  REGION 1..122: note = Synthetic: HC variable region
QVQLVQSGAEVKKPGASVKVSCKASGYTFTSYYIHWVRQAPGQRLEWMGWIFPGSDITKYNEKFKGRVTITRDTSASTAYMELSSLRSEDTAVYYCAREDYGISTYSAMDFWGQGTLVTVSS

SEQ ID NO: 59 - protein, 108 aa, synthetic construct
  REGION 1..108: note = Synthetic: LC variable region
DIQLTQSPSFLSASVGDRVTITCKASQDVGTAVAWYQQKPGKAPKLLIYWASTRHTGVPSRFSGSGSGTEFTLTISSLQPEDFATYYCQQYSSYPLTFGQGTKVEIKR

SEQ ID NO: 60 - protein, 5 aa, synthetic construct
  REGION 1..5: note = Synthetic: HC CDR1 of 18E04
HFDIN

SEQ ID NO: 61 - protein, 17 aa, synthetic construct
  REGION 1..17: note = Synthetic: HC CDR2 of 18E04
WMNPDSDNTDYAQEFQG

SEQ ID NO: 62 - protein, 7 aa, synthetic construct
  REGION 1..7: note = Synthetic: HC CDR3 of 18E04
GGTTLDY

SEQ ID NO: 63 - protein, 11 aa, synthetic construct
  REGION 1..11: note = Synthetic: LC CDR1 of 18E04
SGDALPKKYAY

SEQ ID NO: 64 - protein, 7 aa, synthetic construct
  REGION 1..7: note = Synthetic: LC CDR2 of 18E04
EDSKRPS

SEQ ID NO: 65 - protein, 11 aa, synthetic construct
  REGION 1..11: note = Synthetic: LC CDR3 of 18E04
YSTDSSDNHVI

SEQ ID NO: 66 - protein, 5 aa, synthetic construct
  REGION 1..5: note = Synthetic: HC CDR1 of 98C07
DYYMS

SEQ ID NO: 67 - protein, 16 aa, synthetic construct
  REGION 1..16: note = Synthetic: HC CDR2 of 98C07
YISRSSFIYYSESVKG

SEQ ID NO: 68 - protein, 8 aa, synthetic construct
  REGION 1..8: note = Synthetic: HC CDR3 of 98C07
WELSPFDY

SEQ ID NO: 69 - protein, 11 aa, synthetic construct
  REGION 1..11: note = Synthetic: LC CDR1 of 98C07
RASQGISNYLA

SEQ ID NO: 70 - protein, 7 aa, synthetic construct
  REGION 1..7: note = Synthetic: LC CDR2 of 98C07
AASSLQS

SEQ ID NO: 71 - protein, 9 aa, synthetic construct
  REGION 1..9: note = Synthetic: LC CDR3 of 98C07
QQYNTYPFT

SEQ ID NO: 72 - protein, 5 aa, synthetic construct
  REGION 1..5: note = Synthetic: HC CDR1 of 1C02
YYGIS

SEQ ID NO: 73 - protein, 17 aa, synthetic construct
  REGION 1..17: note = Synthetic: HC CDR2 of 1C02
WISANSGNTNYAQKFQG

SEQ ID NO: 74 - protein, 8 aa, synthetic construct
  REGION 1..8: note = Synthetic: HC CDR3 of 1C02
GGVAVLEY

SEQ ID NO: 75 - protein, 11 aa, synthetic construct
  REGION 1..11: note = Synthetic: LC CDR1 of 1C02
WASQGISSYLA

SEQ ID NO: 76 - protein, 7 aa, synthetic construct
  REGION 1..7: note = Synthetic: LC CDR2 of 1C02
VASTLQS

SEQ ID NO: 77 - protein, 9 aa, synthetic construct
  REGION 1..9: note = Synthetic: LC CDR3 of 1C02
QQLKIYPLT

SEQ ID NO: 78 - protein, 5 aa, synthetic construct
  REGION 1..5: note = Synthetic: HC CDR1 of 1C06
DYYMN

SEQ ID NO: 79 - protein, 17 aa, synthetic construct
  REGION 1..17: note = Synthetic: HC CDR2 of 1C06
DISSRDNTIYYADSVKG

SEQ ID NO: 80 - protein, 14 aa, synthetic construct
  REGION 1..14: note = Synthetic: HC CDR3 of 1C06
ARERGFGDYFGMDV

SEQ ID NO: 81 - protein, 11 aa, synthetic construct
  REGION 1..11: note = Synthetic: LC CDR1 of 1C06
RASQDISSALA

SEQ ID NO: 82 - protein, 7 aa, synthetic construct
  REGION 1..7: note = Synthetic: LC CDR2 of 1C06
DASSLES

SEQ ID NO: 83 - protein, 9 aa, synthetic construct
  REGION 1..9: note = Synthetic: LC CDR3 of 1C06
QQFNTYPLT

SEQ ID NO: 84 - protein, 122 aa, synthetic construct
  REGION 1..122: note = Heavy chain variable region (VH) of antibody F19
QVQLQQWGAGLLKPSETLSLTCAVYGGSFSGYNWHWIRQPPGKGLEWIGEITHSGSTNYNPSLKSRVTISVDTSKNQFSLKLSSVTAADTAVYYCVREIAVAGTGYYGMDVWGQGTTVTVSS

SEQ ID NO: 85 - protein, 107 aa, synthetic construct
  REGION 1..107: note = Light chain variable region (VL) of antibody F19
AIQLTQSPSSLSASVGDRVTITCRASQGINSAFAWYQQKPGKAPKLLIYDASSLESGVPSRFSGSGSGTDFTLTISSLQPEDFATYYCQQFNSYPLTFGGGTKVEIK